(19) United States
(12) Patent Application Publication
(10) Pub. No.: US 2009/0054965 A1
(43) Pub. Date: Feb. 26, 2009

(54) Methods For Producing Embolic Devices

(75) Inventor: Robert Richard, Wrentham, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Boston Scientific Scimed, Inc.

(21) Appl. No.: 12/195,065

(22) Filed: Aug. 20, 2008

Related U.S. Application Data
(60) Provisional application No. 60/957,010, filed on Aug. 21, 2007.

(51) Int. Cl. A61F 2/06

(52) U.S. Cl. 623/1.15; 623/1.44; 623/1.49

(57) ABSTRACT

A vascular implant is provided. The implant can comprise a first material layer and at least one metallic material disposed on at least a portion of the first material layer in a predetermined pattern. The implant can further comprise at least one hydrophobic material disposed on at least a portion of the surface of at least one of the first material layer and the at least one metallic material.

The present disclosure claims priority under 35 U.S.C. §119 to U.S. Provisional Patent Application 60/957,010, which is also hereby expressly incorporated by reference in its entirety.

### TECHNICAL FIELD

The present disclosure is directed towards methods for producing embolization devices and to devices produced using these methods.

### BACKGROUND

Embolization systems have become an important clinical tool for treating a variety of medical problems. For example, embolic devices can be used to stop or limit blood flow to tumors, thereby potentially destroying part or all of the tumor, or shrinking the tumor in preparation for or in conjunction with surgery or other treatments (e.g. chemotherapy). In addition, embolic devices may be used to treat bleeding, as caused by, for example, vascular malformations or uterine fibroids.

Production of embolic devices having suitable dimensions can be difficult. For example, for some clinical conditions, it may be desirable to produce small coils that can be delivered to comparably-sized anatomic sites. Further, in some cases, the coil must be delivered through a small catheter lumen in a partially-coiled or uncoiled shape (i.e. as a linear strand or fiber), and after the device is properly positioned, the coil must be twisted to form a space-occupying three-dimensional structure. Production of coils that can be delivered through small lumens and made to form desired three-dimensional structures can be difficult and/or expensive.

In addition, in some cases, it may be desirable to have embolic devices available that can carry and/or deliver therapeutic agents to selected anatomic sites. For example, suitable therapeutic agents can include chemotherapeutic agents, antibiotics, and/or drugs that affect thrombosis (e.g. procoagulants, anti-coagulants, and/or drugs that affect platelet aggregation). Further, it may be desirable to control drug delivery so that certain drugs are released only at selected anatomic sites after implantation of the embolic device.

The present disclosure is directed at methods of producing embolic devices that can be delivered to an anatomic site and made to produce a desired three-dimensional structure and/or release therapeutic agents after delivery of the device. The present disclosure is further directed towards devices that may be produced by the disclosed methods.

### SUMMARY

A first aspect of the present disclosure includes a vascular implant. The implant can comprise a first material layer and at least one metallic material disposed on at least a portion of the first material layer in a predetermined pattern. The implant can further comprise at least one hydrophobic material disposed on at least a portion of the surface of at least one of the first material layer and the at least one metallic material.

A second aspect of the present disclosure includes a method of producing a vascular implant. The method can include selecting a first material layer and applying at least one metallic material to at least a portion of the first material layer in a predetermined pattern. The method can further include applying at least one hydrophobic material to at least a portion of the surface of at least one of the first material layer and the at least one metallic material.

A third aspect of the present disclosure includes a vascular embolization system. The system can comprise a catheter configured to be inserted into a vein or artery and having an elongate passage with an opening configured to be positioned at a selected anatomic site within a vascular structure. The system can further include a vascular implant having an elongate shape. The implant can comprise a first material layer and at least one metallic material disposed on at least a portion of the first material layer in a predetermined pattern. The implant can further comprise at least one hydrophobic material disposed on at least a portion of the surface of at least one of the first material layer and the at least one metallic material.

A fourth aspect of the present disclosure comprises a method of implanting a medical device within a vascular structure. The method can include positioning a catheter within a vein or artery and advancing at least a portion of the catheter to a selected anatomic site. The method can further include selecting an implant comprising a first material layer and at least one metallic material disposed on at least a portion of the first material layer in a predetermined pattern. The implant can further comprise at least one hydrophobic material disposed on at least a portion of the surface of at least one of the first material layer and the at least one metallic material. The implant can be formed into a predetermined three-dimensional structure.

Additional aspects and advantages of the invention will be set forth in part in the description which follows, and in part will be apparent from the description, or can be learned by practice of the invention. The advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1A illustrates an embolic coil and vascular delivery system before delivery to a selected anatomic site, according to an exemplary disclosed embodiment.

FIG. 1B illustrates another embolic coil that may be delivered to a selected anatomic site, according to an exemplary disclosed embodiment.

FIG. 2A illustrates an embolic coil being delivered to a selected anatomic site, according to an exemplary disclosed embodiment.

FIG. 2B illustrates another embolic coil in a three-dimensional configuration that may be formed by the coil after deployment within a selected anatomic site.

FIG. 2C illustrates another embolic coil in a three-dimensional configuration that may be formed by the coil after deployment within a selected anatomic site.

FIG. 3 illustrates a process for producing embolic devices, according to exemplary embodiments of the present disclosure.

### DESCRIPTION OF THE EMBODIMENTS

Reference will now be made in detail to the exemplary embodiments of the invention, examples of which are illustrated in the accompanying drawings. Wherever possible, the same reference numbers will be used throughout the drawings to refer to the same or like parts.

The embolic devices of the present disclosure may be referred to as “coils” or “devices.” It will be understood that “coil” can refer to any device that can be deployed in a body and may be made to alter its shape according to the methods and devices of the present disclosure. Further, it will be understood that such changes to produce a “coil” or “device can include twisting, folding, curling, and/or any other shape-altering process.

Typical vascular embolization systems include beads or coils that are released into the vasculature to block blood flow and cause thrombosis. Generally, such systems are simply pushed or otherwise forced through a conduit. In the case of vascular coils, the coils are held in a relatively elongate configuration by the geometric constraints of the delivery system, and when pushed out of the delivery system, may assume a desired three-dimensional structure based on spring-like properties of the materials from which they are formed.

However, it can be difficult to produce smaller coils having desired three-dimensional structures after delivery to selected anatomic sites. The present disclosure provides vascular coils that can be produced with a wide range of sizes. Further, the coils of the present disclosure can include an active folding mechanism, whereby the surface tension on various patterned layers causes the coils to fold into predetermined three-dimensional structures when exposed to a hydrophilic environment such as blood.

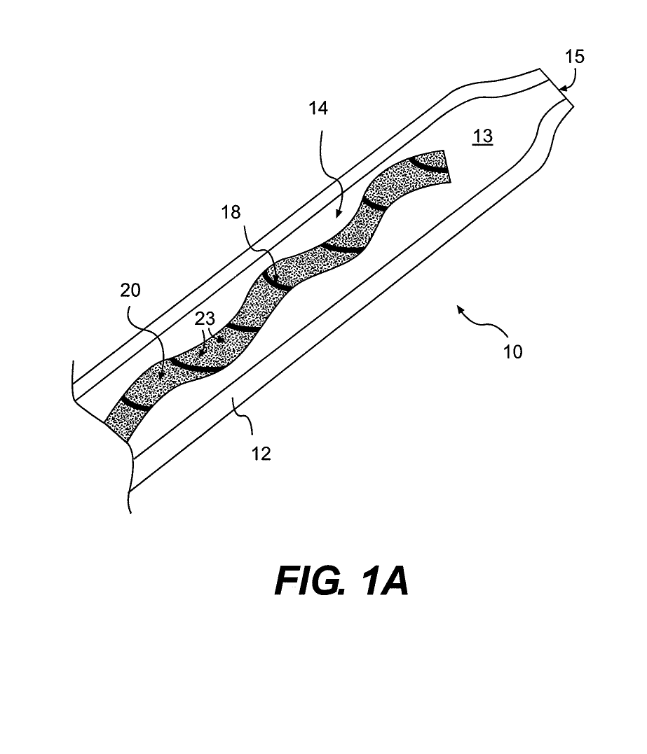

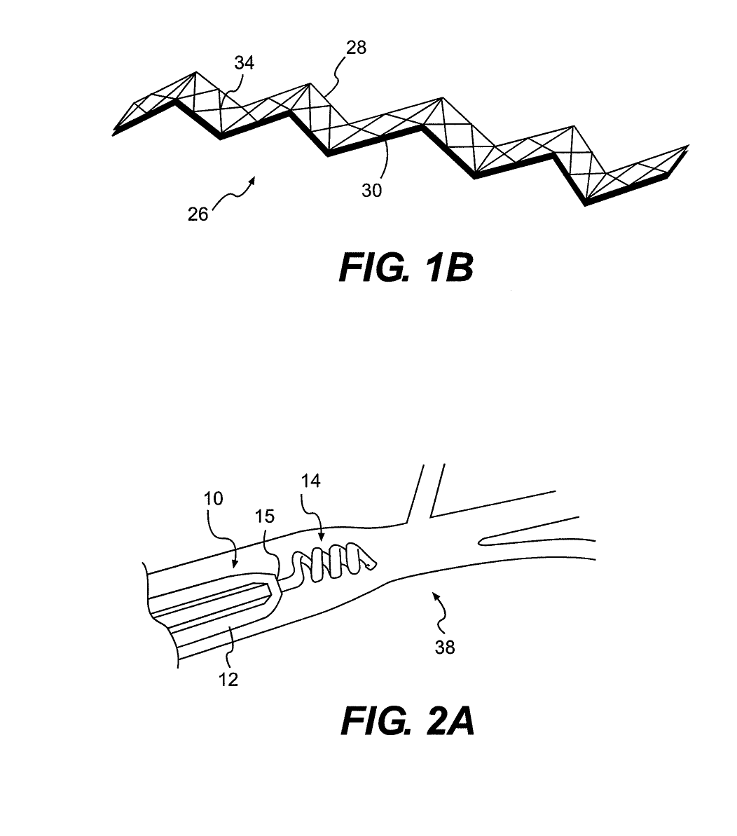

FIG. 1A illustrates an embolic coil 14 and vascular delivery system 10 before delivery to a selected anatomic site. As shown, coil 14 includes an elongated structure configured to be positioned within an inner passageway 13 of a sheath or vascular catheter 12. When the sheath or catheter 12 of vascular delivery system 10 is positioned at a desired anatomic site, coil 14 can be deployed at the selected site by pushing or otherwise moving coil 14 through an opening 15 at a distal tip of sheath or catheter 12. In some embodiments, coil 14 may be configured to produce a desired three-dimensional structure after being deployed through opening 15. Further, coil 14 can be configured to produce the desired three-dimensional structure when exposed to a hydrophilic environment and/or by application of an electric current to one or more regions of coil 14.

As shown, coil 14 can include a number of components. For example, as shown, coil 14 includes a first layer of material 20, which forms a substrate for coil 14. Further, first layer 20 can include a second material 18, which may be applied in a pattern configured to produce a desired three-dimensional structure when coil 14 is positioned at a desired anatomic site, as described in detail below. In addition, a coating material 23 may be applied to all or part of the surfaces of first layer 20 and/or second material 18.

First layer 20 can be produced from a variety of suitable materials. For example, a number of suitable biodegradable and non-biodegradable polymers may be selected for first layer 20. The materials used to produce first layer 20 may be selected based on a variety of factors. For example, suitable materials may be selected based on desired mechanical and/or biological properties. For example, suitable materials may be selected to produce a desired degree of rigidity and/or strength to facilitate passage of coil 14 through delivery system 10. In some embodiments, layer 20 may be produced from materials having sufficient rigidity and/or strength to allow coil 14 to be pushed through passage 13, while allowing coil 14 to produce a desired three-dimensional structure when in a desired anatomic site.

In addition, suitable materials may be selected to facilitate certain treatments or biological processes. For example, biodegradable materials may be selected, and such materials may incorporate one or more drugs or therapeutic agents. For example, suitable drugs can include chemotherapeutic drugs, anticoagulants, procoagulants (e.g. thrombin or any enzymes facilitating formation of thrombin), drugs affecting platelet aggregation, and/or antibiotics. Further, biodegradable materials may be selected to allow temporary treatment by temporary vascular occlusion or drug delivery.

In addition, to facilitate thrombosis and more complete blockage of a blood vessel, suitable coils can include additional features or surface modifications selected to enhance thrombosis. For example, coils can include a number of fibers produced from materials that enhance thrombosis. Further, coils can include prothrombotic agents applied to their surface or embedded within the coil materials.

A variety of suitable non-biodegradable materials and biodegradable materials can be selected for first layer 20. Suitable non-biodegradable polymers can include include polystrene; polyisobutylene copolymers and styrene-isobutylene-styrene block copolymers (e.g. styrene-isobutylene-styrene tert-block copolymers); polyvinylpyrrolidone; cross-linked polyvinylpyrrolidone; polyvinyl alcohols, copolymers of vinyl monomers; polyvinyl ethers; polyvinyl aromatics; polyethylene oxides; polyesters including polyethylene terephthalate; polyamides; polyacrylamides; polyethers (e.g. polyether sulfone); polyalkylenes (e.g. polypropylene); polyethylene; high molecular weight polyethylene; polyurethanes; polycarbonates; polyetheretherketones, polyetherketones; polyethylene terephthalate; polybutylene terephthalate; polyphenylene sulfide; polyphenylene oxide; and/or polyphosphazenes. Non-limiting examples of suitable biodegradable polymers can include polycarboxylic acid; polyanhydrides (e.g. maleic anhydride polymers); polyorthoesters; poly-amino acids; polyethylene oxide; polyphosphazenes; polylactic acid; polyglycolic acid; poly(L-lactic acid); poly(D,L,-lactide); poly(lactic acid-co-glycolic acid); 50/50 (DL-lactide-co-glycolide); polydioxanone; polypropylene fumarate; polydepsipeptides; polycaprolactone and co-polymers and mixtures thereof (e.g. poly(D,L-lactide-co-caprolactone) and polycaprolactone co-butylacrylate); polyhydroxybutyrate valerate and blends; polycarbonates (e.g. tyrosine-derived polycarbonates and arylates, polyiminocarbonates, and polydimethyltrimethylcarbonates); cyanoacrylate; calcium phosphates; polyglycosaminoglycans; macromolecules (e.g. polysaccharides such as hyaluronic acid); cellulose; hydroxypropylmethyl cellulose; gelatin; starches; dextrans; alginates; proteins and polypeptides; and/or mixtures and copolymers of any of the foregoing.

In addition, second material 18 can include a number of suitable materials. For example, in some embodiments, second material 18 can include one or more electroactive metals. For example, suitable electroactive metals can include a variety of suitable biologically inert materials such as platinum or gold, which may be selected based on cost, manufacturability, and/or biological effect.

As noted previously, part or all of coil 14 may be covered with a coating material 23. Coating material 23 may be selected such that application of an electrical current will cause coating material 23 to coalesce or otherwise produce a different shape, thereby causing coil 14 to fold into a desired three-dimensional structure. Alternatively or additionally, coating material 23 may be selected such that coating material 23 will coalesce when exposed to a hydrophilic environment such as blood. In some embodiments, coating material 23 will include a conducting polymer, hydrophobic materials, or conducting and hydrophobic materials. For example, suitable polymers for coating material 23 can include liquid crystal polymers, long chain poly (meth)acrylkates, conducting polymers (polyanalines, polypyroles), low molecular weight waxy polymers, amphiphilic block copolymers.

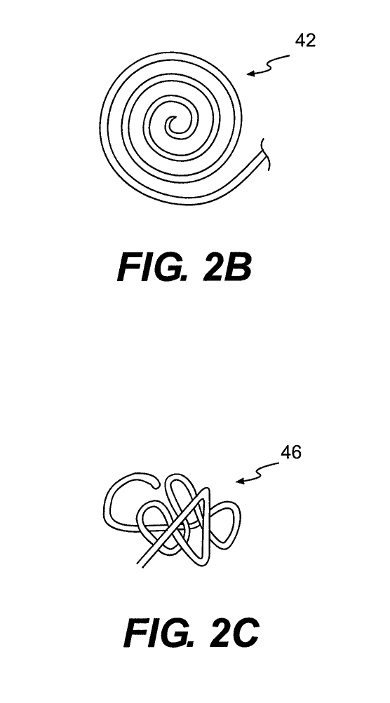

As noted previously, second material 18 may be applied in a pattern configured to cause coil 14 to fold into a variety of desired three-dimensional shapes. For example, as shown in FIG. 1A, second material 18 may be applied as elongate strands diagonally crossing first layer 20. This configuration may be suitable for producing a coiled shape (as shown in FIG. 2A). However, a variety of other suitable patterns may be selected.

FIG. 1B illustrates another embolic coil that may be delivered to a selected anatomic site, according to an exemplary disclosed embodiment. In this embodiment, coil 26 includes a first layer 28 and second layer 30. First layer 28 and second layer 30 may be produced from various materials, as described above with reference to first layer 20. In addition, coil 26 can include a second material 34 and a coating material (not shown) covering all or part of the surface of coil 26. As shown, second material 34 can be applied in a predetermined pattern, such as a crossing pattern. Such a configuration may be selected to produce a coil 14 that will fold into a star-like shape.

In the embodiment of FIG. 1B, coil 26 includes two layers 28, 30, which may be attached to one another. The use of two or more layers may be desirable to produce coils having certain mechanical and/or biological properties. For example, in some embodiments, either or both of layers 28, 30 may be produced from a material having a desired strength or rigidity to prevent coil buckling within a catheter or sheath. Further, at least one layer may be produced from a material having a sufficient bondability with a selected second material to be applied in a desired pattern. In addition, layers 28, 30 may be produced from materials having different degrees of hydrophobicity to facilitate folding to produce a desired three-dimensional structure.

As noted, selected coils may be delivered to desired anatomic sites, and once positioned in a desired site, one or more coils may be caused to fold into a selected three-dimensional structure. FIG. 2A illustrates an embolic coil 14 being delivered to a selected anatomic site, according to an exemplary disclosed embodiment. As shown, coil 14 is delivered through a blood vessel 38 (e.g. an artery or vein) using a catheter or sheath 12. Once located at the desired site, coil 14 is advanced through opening 15 of catheter or sheath 12 and caused to form a desired three-dimensional structure by application of a current and/or exposure to a hydrophilic environment.

Before or after forming the desired three-dimensional shape, and once properly positioned, coil 14 may be detached so that sheath or catheter 12 can be removed and coil 14 left in place. Those skilled in the art will appreciate that coil 14 can be detached using a number of suitable mechanisms, including any suitable mechanical, electrolytic, and/or chemical detachment mechanism. Further, in some embodiments, coil 14 may be advanced completely through opening 15, thereby causing coil 14 to be released.

As noted, the desired anatomic site may be selected to facilitate a variety of suitable medical treatments. For example, as shown, coil 14 may be positioned within vessel 38 to partially or completely block blood flow through one or more vessels downstream of coil 14. In some embodiments, coil 14 may be positioned upstream of a tumor. In other embodiments, coil 14 may be positioned within a selected blood vessel to limit blood flow in order to treat a vascular malformation, a fibroid, any benign or malignant growth, to treat bleeding from a tissue or organ site, to treat a vascular aneurysm, or to destroy or limit blood flow to a tissue or organ that is to be removed by surgery. Further, in other embodiments, as noted above, coil 14 can include one or more therapeutic agents to facilitate treatment and/or prevent side effects.

During coil implantation, a physician will generally visualize the position of the coil and/or surrounding anatomic structures using one or more known visualization systems. Such systems can include, for example, fluoroscopy, CT scanning, ultrasound, MRI, and/or any other suitable visualization system. In some embodiments, coil 14 may include one or more radio-opacifying agents within its structure to facilitate viewing during insertion and at any point while the device is implanted. Non-limiting examples of radio-opacifying agents are bismuth subcarbonate, bismuth oxychloride, bismuth trioxide, barium sulfate, tungsten, and mixtures thereof. These agents may be embedded within one or more layers 28, 30 of coil.

As noted, selected coils may be configured to form a variety of configurations after positioning within a desired anatomic site. The specific configuration can be selected based on the desired clinical outcome, manufacturability, and/or physician preference. For example, various shapes may be selected to control overall surface area in order to affect the rate of drug release, biodegradation, and/or thrombosis. Additionally, specific coil sizes and shapes may be selected to facilitate occlusion of various blood vessels. As noted previously, and as shown in FIG. 2A, coil 14 can include a substantially spiral shape. Alternatively, FIG. 2B illustrates another embolic coil 42 in a three-dimensional configuration that may be formed by the coil after deployment within a selected anatomic site. This coil 42 includes a planar spiral shape. Further, FIG. 2C illustrates another embolic coil 46 in a three-dimensional configuration that may be formed by coil 46 after deployment within a selected anatomic site. This shape includes a substantially globular shape that may be selected to fill an aneurysm or occlude a selected vessel.

The coils of the present disclosure can be produced using a number of processes and materials. Further, suitable coils can be produced having a range of sizes and shapes. For example, suitable coils can be produced having a substantially elongate configuration before implantation, as shown in FIG. 1A, and having a width configured to allow the coils to be passed through an elongate passage of a catheter or sheath. A range of suitable sizes may be selected depending on the anatomic site to be treated. For example, suitable coils can have a width between about 0.5 mm and about 2 mm, but it will be understood that larger or smaller devices may be selected depending on the site to be treated.

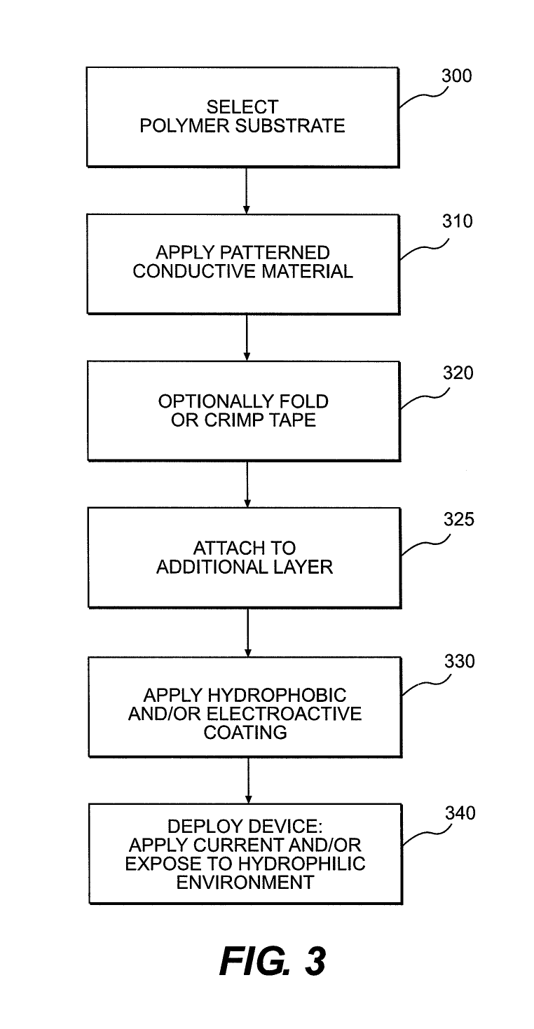

FIG. 3 illustrates a process for producing embolic devices, according to exemplary embodiments of the present disclosure. As shown at Step 300, a first substrate material is first selected. As noted above, a variety of suitable materials can be selected, which are then cut or otherwise formed into a desired shape. For example, as shown in FIG. 1A, coil first layer 20 includes an elongated tape. However, a variety of shapes may be selected. For example, in some embodiments, suitable coils will have a number of possible elongated configurations that can be folded into desired three-dimensional structures. For example, instead of a flat, elongated tape, the substrate can be substantially cylindrical, have varying thicknesses, or have surface textures configured to facilitate bonding to other coil materials or to promote thrombosis.

Next, as shown at Step 310, at least one second material is applied to the first substrate layer. As noted, such materials may be applied in a range of patterns selected to facilitate formation of a desired shape after exposure to a hydrophilic environment and/or on application of an electric current. In addition, to produce desired patterns and small dimensions desired for smaller coils, a variety of pattern deposition processes can be used. For example, suitable materials may be applied using a number of electrical materials processing techniques, including for example, photolithography, photolithography with etching (e.g. plasma or chemical etching), chemical vapor deposition processes, physical vapor deposition processes, hybrid processes, and/or any combinations of known materials deposition processes.

Next, as shown at Step 320, the first layer and second material may be optionally crimped or folded. Crimping or folding can facilitate attachment of the first layer to a second layer, or an additional layer, as shown at Step 325. As noted, a second layer 30 can optionally be attached to first layer 28 (as described with reference to FIG. 1B) and can be made from a variety of suitable materials. Further, first layer 28 and second layer 30 may be produced from similar materials or from different materials, and the specific materials can be selected based on desired physical properties of the completed coils, and/or to facilitate formation of a desired three-dimensional structure in a blood vessel.

Further, as shown at Step 330, hydrophobic and/or electroactive materials can be applied to all or part of the surface of coil 14. In some embodiments, the hydrophobic or electroactive material can be applied to only selected regions of coil 14. For example, electroactive materials may be applied only around or on portions of a second material 18 that is configured to carry an electrical current through the material, thereby affecting the electroactive coating and causing the coil to fold into a desired three-dimensional shape.

As noted previously, completed coils can be implanted using a variety of suitable vascular delivery systems configured to access various arteries and/or veins depending on the anatomic site to be treated. Once a coil is positioned at the desired anatomic site, the coil can be made to fold into a selected three-dimensional configuration, as shown at Step 340, by application of electrical current and/or exposure to a hydrophilic environment. In some embodiments, application of an electrical current will cause a conformational change in an electroactive material. In other embodiments, application of a current will heat the coil, thereby facilitating formation of a desired three-dimensional shape.

It should be noted that selected coils may be produced as prepackaged products with selected delivery systems and/or as individual items. For example, in some embodiments, it may be desirable for a physician to insert a catheter or sheath into the vasculature of a patient wherein a coil to be delivered is contained within the catheter or sheath at the time of insertion. In other embodiments, the physician may insert the catheter or sheath and later advance a coil through the catheter or sheath once the catheter or sheath is positioned at a desired site. Further, in some embodiments, a catheter may be positioned within the vasculature, and a separate catheter or sheath containing a coil may be advanced through the catheter to the selected anatomic site.

Exemplary embodiments of the present invention have been described above. Those skilled in the art will understand, however, that changes and modifications may be made to these embodiments without departing from the true scope and spirit of the invention, which is defined by the claims.

1. A vascular implant, comprising:
a first material layer;
at least one metallic material disposed on at least a portion of the first material layer in a predetermined pattern; and
at least one hydrophobic material disposed on at least a portion of the surface of at least one of the first material layer and the at least one metallic material.

2. The implant of claim 1, wherein the at least one metallic material includes an electroactive metal.

3. The implant of claim 1, wherein the implant is configured to change shape when exposed to an electrical current.

4. The implant of claim 1, wherein the implant is configured to change shape when exposed to a hydrophilic environment.

5. The implant of claim 4, wherein the hydrophilic environment includes blood.

6. The implant of claim 1, further including a second material layer covering at least a portion of the first material layer.

7. A method of producing a vascular implant, comprising:
selecting a first material layer;
applying at least one metallic material to at least a portion of the first material layer in a predetermined pattern; and
applying at least one hydrophobic material to at least a portion of the surface of at least one of the first material layer and the at least one metallic material.

8. The method of claim 7, wherein the at least one metallic material includes an electroactive metal.

9. The method of claim 7, wherein the implant is configured to change shape when exposed to an electrical current.

10. The method of claim 7, wherein the implant is configured to change shape when exposed to a hydrophilic environment.

11. The method of claim 10, wherein the hydrophilic environment includes blood.

12. The method of claim 7, further including attaching a second material layer to the first material layer.

13. A vascular embolization system, comprising:
a catheter configured to be inserted into a vein or artery; and
a vascular implant having a substantially linear shape, comprising:
a first material layer;
at least one metallic material disposed on at least a portion of the first material layer in a predetermined pattern; and
at least one hydrophobic material disposed on at least a portion of the surface of at least one of the first material layer and the at least one metallic material.

14. The system of claim 13, wherein the at least one metallic material includes an electroactive metal.

15. The system of claim 13, wherein the implant is configured to change shape when exposed to an electrical current.

16. The system of claim 13, wherein the implant is configured to change shape when exposed to a hydrophilic environment.

17. The system of claim 16, wherein the hydrophilic environment includes blood.

18. The system of claim 13, wherein the implant further includes a second material layer attached to the first material layer.

19. A method of implanting a medical device within a vascular structure, comprising:
selecting an implant comprising:
a first material layer;
at least one metallic material disposed on at least a portion of the first material layer in a predetermined pattern; and
at least one hydrophobic material disposed on at least a portion of the surface of at least one of the first material layer and the at least one metallic material;
inserting a catheter within a vein or artery;
advancing at least a portion of the catheter to a selected anatomic site;
extending at least a portion of the implant into a vascular structure proximate the selected anatomic site; and
causing the implant to form a predetermined three-dimensional structure.

20. The method of claim 19, wherein causing the implant to form a predetermined three-dimensional structure includes applying an electrical current to the implant.

21. The method of claim 19, wherein causing the implant to form a predetermined three-dimensional structure includes exposing the implant to blood.

22. The method of claim 19, wherein the implant further includes a second material layer attached to the first material layer.
